(19) United States
(12) Plant Patent Application Publication
(10) Pub. No.: US 2005/0066398 P1
(43) Pub. Date: Mar. 24, 2005

(54) Rose plant named 'Poulasor'

(76) Inventor: Mogens N. Olesen, Fredensborg (DK)

Correspondence Address: Poulsen Roser Pacific, Inc., 620 South Front Street, Central Point, OR, 97502, US

(21) Appl. No.: 10/950,227

(22) Filed: Sep. 23, 2004

Related U.S. Application Data
(63) Continuation of application No. 10/339,871, filed on Jan. 10, 2003.

(51) Int. Cl. A01H 5/00

(52) U.S. Cl. PLT/148

(57) ABSTRACT

A new floribunda, garden rose plant with attractive foliage and abundant pink flowers. This new and distinct variety has shown to be uniform and stable in the resulting generations from asexual propagation.

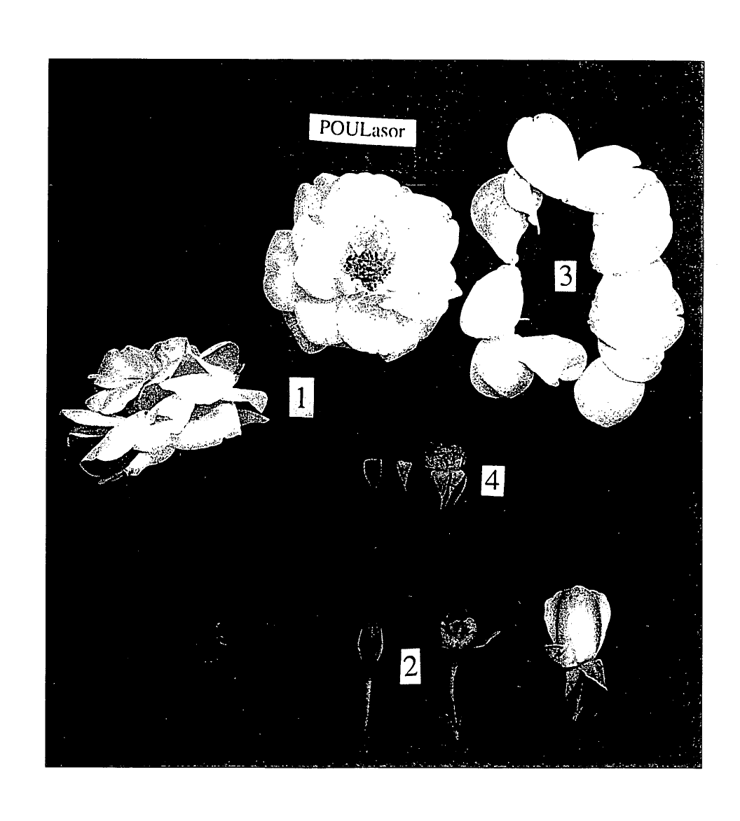

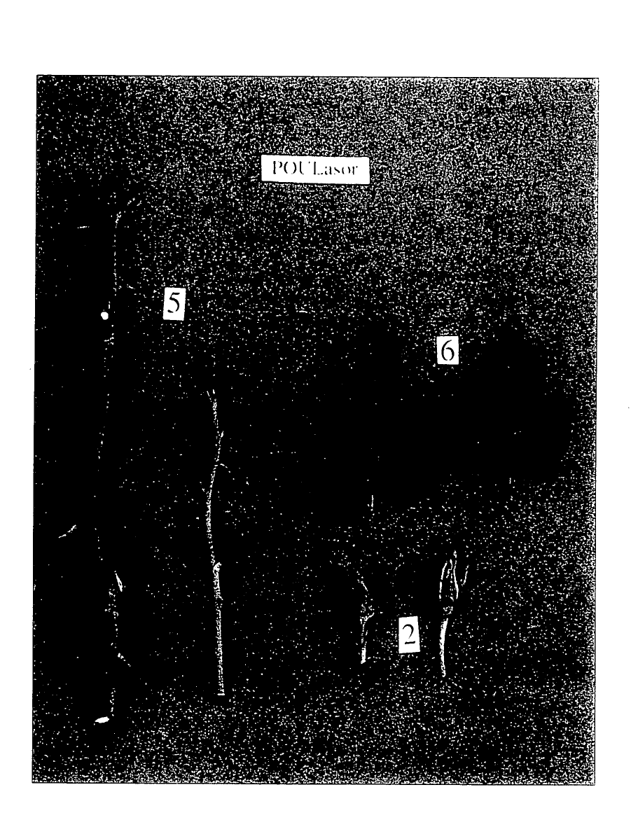

This application is a continuation of U.S. Plant patent application Ser. No. 10/339,871 filed on Jan. 10, 2003.

### SUMMARY OF THE INVENTION

Classification:

Botanical: Rosa hybrid.

Commercial: Floribunda.

The present invention constitutes a new and distinct variety of garden rose plant which originated from a controlled crossing between ‘Poulrine’, non-patented, and ‘Poulskov’ described and illustrated in U.S. Plant Pat. No. 9,062, issued on Feb. 28, 1995. The two parents were crossed during the summer of 1991 and the resulting seeds were planted in a controlled environment in Fredensborg, Denmark. The new variety is named ‘Poulasor’.

The new rose may be distinguished from its pollen parent, ‘Poulskov’, by the following combination of characteristics:
1. The pollen parent has a petal count of 48 to 62 petals, while the applicant variety has 25 to 27 petals.
2. The pollen parent has a bud color of between Red Group 55D and Red Group 56A, while ‘Poulasor’ has a bud color of Red Group 55D.

The new variety may be distinguished from its seed parent, ‘Poulrine’, described in patent application Ser. No. 09/287,292, now abandoned, by the following combination of characteristics:
1. ‘Poulrine’ has a petal count of 18 to 22 petals, while ‘Poulasor’ has a petal count of 25 to 27 petals.
2. ‘Poulrine’ has a bud color of Red-Purple Group 57D, while ‘Poulasor has a bud color of Red Group 55D.

The objective of the hybridization of this rose variety was to create a new and distinct variety for garden use with unique qualities, such as:
1. Uniform and abundant flowers;
2. Vigorous, but compact growth when propagated both as a budded rose and on its own roots;
3. Disease resistance.

This combination of qualities is not present in previously available commercial cultivars of this type known to the inventors, and distinguish ‘Poulasor’ from all other varieties of which we are aware.

As part of the rose development program, Mogens N. Olesen germinated the seeds from the aforementioned hybridization during winter 1991 and conducted evaluations on the resulting seedlings in a controlled environment in Fredensborg, Denmark.

‘Poulasor’ was selected in the spring 1992 by the inventors as a single plant from the progeny of the aforementioned hybridization.

Asexual reproduction of ‘Poulasor’ by traditional budding and rooted cuttings was first done by Mogens N. Olesen in the nursery in Fredensborg, Denmark in August, 1992. This initial and other subsequent asexual propagations conducted in controlled environments have demonstrated that the characteristics of ‘Poulasor’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulasor’. Specifically illustrated in the drawing:
1. Stem showing branching and the attachment of leaves, buds, and peduncles;
2. Flower bud, partially opened bud, and open bloom;
3. Flower petals, detached;
4. Sepals, receptacle, and pedicel;
5. Stem as well as a bare stem exhibiting thorns;
6. Leaves.

### DETAILED DESCRIPTION OF THE VARIETY

The following is a description of ‘Poulasor’, as observed in its growth in a field nursery in Jackson County, Oreg. Observed plants are 3 years of age. Color references are made using The Royal Horticultural Society (London, England) Colour Chart, 1995, except where common terms of color are used.

For a comparison, several physical characteristics of the rose variety ‘Poulskov’, a rose variety from the same inventors described and illustrated in U.S. Plant Pat. No. 9,062 and issued on 28 Feb., 1995 are compared to ‘Poulasor’ in Chart 1.

| | ‘Poulasor’ | ‘Poulskov’ |
|---|---|---|
| Petal Count | 25 to 27. | 48 to 62. |
| Bud color | Red Group 55D. | Between Red Group |
| | | 55D and Red Group |
| | | 56A. |
| Thorn color | Yellow-Green Group | Greyed-Orange Group |
| | 145C. | 165B. |

Parents:
   Seed parent.—‘Poulrine’.
   Pollen parent.—‘Poulskov’.

### FLOWER AND FLOWER BUD

Blooming habit: Continuous.
Flower bud:
   Size.—Upon opening, 30 mm in length from base of receptacle to end of bud. Typical and observed bud diameter is 12 mm.
   Bud form.—Long, pointed ovoid.
   Bud color.—As sepals unfold, Red Group 55C. At ¼ opening, Red Group 55C.
   Sepals.—Upper surface is Yellow-Green Group 144B in interior, Yellow-Green Group 144A at margins. Lower surface is Yellow-Green Group 144B in interior, Yellow-Green Group 144A at margins. Weak foliaceous appendages on three of the five sepals. Surfaces of sepals moderately pubescent. Stipitate glands are present along the margins of the sepals. Shape: Overall shape of sepals is lanceolate. The sepal apex is aristate. Margins are entire with medium foliaceous appendages. Base is flat at union with receptacle. Size: 25 to 30 mm (l)×8 mm (w).
   Receptacle.—Surface: Moderate amounts of fragrant stipitate glands present. Shape: Funnel shaped. Size: 6 mm (h)×5 mm (w). Color: Yellow-Green Group 144A.
   Peduncle.—Surface: Moderately rough due to the presence of stipitate glands. Diameter: 2 mm. Length: 25 to 30 mm average length. Color: Yellow-Green Group 144A. Strength: Somewhat strong.
   Borne.—3 to 4 buds per stem.
Flower bloom:
   Fragrance.—Moderate; floral.
   Duration.—The blooms have a duration on the plant of approximately 7 to 10 days. Petals fall cleanly away from plant.
   Size.—Average flower diameter is 55 mm when open. Flower depth is 30 mm.
   Form.—Shape of flower when viewed from the side: Upon opening, upper part:Convex. Upon opening, lower part:Flat. Open flower, upper part: Convex. Open flower, lower part: Concave.
   Petalage.—Very double. Average range: 25 to 27 petals under normal conditions with 1 to 3 petaloids.
Color:
   Upon opening, petals.—Outermost petals: Outer side: Red-Purple Group 62D. Inner Side: Red Group 55D. Innermost petals: Outer side: Red-Purple Group 62D. Inner Side: Red Group 55D.
   Upon opening, basal petal spots.—Outermost petals: Outer side: Yellow Group 5C and 5D. Inner Side: Yellow Group 5C and 5D. Innermost petals: Outer side: Yellow Group 5C and 5D. Side: Yellow Group 5C and 5D.
   After opening, petals.—Outermost petals: Outer side: Red-Purple Group 62D. Inner Side: Red Group 55D. Innermost petals: Outer side: Red-Purple Group 62D. Inner Side: Red Group 55D.
   After opening basal petal spots.—Outermost petals: Outer side: Yellow Group 5C and 5D. Inner Side: Yellow Group 5C and 5D. Innermost petals: Outer side: Yellow Group 5C and 5D. Inner Side: Yellow Group 5C and 5D.
General tonality: On open flower Red-Purple Group 62D. No change in the general tonality at the end of the 7th day. Afterwards, general tonality is Red-Purple Group 62C.
Petals:
   Petal reflex.—Slight.
   Petal edge.—Entire, with notch in center of margin.
   Shape.—Deltoid.
   Length.—36 mm.
   Width.—35 mm.
   Texture.—Smooth.
   Thickness.—Average.
   Arrangement.—Not formal.
Petaloids:
   Quantity.—1 to 3.
   Shape.—Elliptic.
   Length.—20 mm.
   Width.—10 mm.
   Color.—Inner and outer sides are Yellow Group 5C and 5D.
Reproductive organs:
   Pistils.—Length: 14 mm long. Quantity: 16 to 20. Color: Yellow-White Group 158B.
   Pollen.—Color: Yellow Group 13A. Quantity: Scant.
   Anthers.—Size: 2 mm long. Color: Yellow Group 11B. Quantity: 35 to 40.
   Filaments.—Color: Yellow Group 5A. Length: 5 to 8 mm.
   Stigmas.—Superior in location to anthers. Color: White Group 155C.
   Styles.—Color: Yellow-Green Group 150D at base, Red-Purple Group 67B below stigma.
   Hips.—None observed.

### PLANT

Plant growth: Bushy. When grown as a budded field grown plant on Rosa multiflora understock, the average height of the plant is 70 cm and the average width is 60 cm.
Stems:
   Color.—Young wood: Yellow-Green Group 144A. Older wood: Yellow-Green Group 144A.
   Diameter.—6 to 8 mm.
   Length.—30 cm.
   Internode.—40 mm between nodes.
   Thorns.—Incidence: 3 to 5 thorns per 10 cm of stem. Size: Average length: 9 mm. Color: Yellow-Green Group 144C. Shape: Upper side is linear; lower side is concave.
   Surface.—Young wood: Rough. Older wood: Rough.
Plant foliage: Normal number of leaflets on leaves in middle of the stem: 5 leaflets.
   Leaf size.—105 mm (l)×73 mm (w).
   Quantity.—2 leaves per every 10 cm of stem.
   Color.—Mature Foliage: Upper Leaf Surface: Yellow-Green Group 147A. Lower Leaf Surface: Yellow-Green Group 147B. Juvenile foliage: Upper Leaf Surface: Green Group 137B. Lower Leaf Surface: Yellow-Green Group 147B. Anthocyanin intonation: Location: New growth of top leaves, rachis, petiole and stipule. Color: Greyed-Red Group 181A.
Plant leaves and leaflets:
   Stipules.—Size: 14 mm (l)×5 mm (w). Quantity: 2 stipules per leaf. Shape: Linear with outward extending apecies. Margin: Entire with stipitate glands along the margin. Color: Yellow-Green Group 144A at margins, Yellow-Green Group 144C in interior.
   Petiole.—Length: 6 to 10 mm long. Diameter: 1.5 mm. Color: Yellow-Green Group 144C. Underneath: Yellow-Green Group 144B and C. With one thorn. Margins: Yellow-Green Group 144A.
   Rachis.—Length: 6 to 10 mm long. Color: Yellow-Green Group 144C. Underneath: Yellow-Green Group 144B and C. With one thorn. Margins: Yellow-Green Group 144A.
   Leaflet.—Size: 63 mm (l)×42 mm (w). Edge: Serrated. Overall shape: Rounded/Ovate. Base shape: Round. Apex shape: Acute. Texture: Thick. Glossiness: Moderate. Arrangement: Odd pinnate. Venation: Reticulate. Vein color: Greyed-Yellow Group 160C.
Disease resistance: Above average resistance to mildew, rust, black spot, and Botrytis under normal growing conditions in Jackson County, Oregon.
Cold hardiness: The variety ‘Poulasor’ has been found to be cold hardy in Fredensborg, Denmark and Jackson County, Oregon.

1. A new and distinct variety of rose plant named ‘Poulasor’ of the floribunda rose class, illustrated and described herein as a distinct and novel rose variety due to its abundant Red Group 55C flowers, vigorous and compact growth, disease resistance, suitability for production from softwood cuttings, durability and extended period of bloom.
